(19) United States
(12) Patent Application Publication
(10) Pub. No.: US 2007/0142667 A1
(43) Pub. Date: Jun. 21, 2007

(54) Process for preparing(+)-2-(4-chlorophenyl)-3-methyl butanoic acid

(76) Inventors: Vaddu Venkata Narayana Reddy, Andhra Pradesh (IN); Khwaja Ishratullah, Andhra Pradesh (IN); Penumacha Venkata Krishnam Raju, Andhra Pradesh (IN); Bhimadaka China Raju, Andhra Pradesh (IN); Attaluri Narasimha Rao, Andhra Pradesh (IN); Tella Ramesh Babu, Andhra Pradesh (IN)

Correspondence Address: LADAS & PARRY, 26 WEST 61ST STREET, NEW YORK, NY, 10023, US

(21) Appl. No.: 10/541,344

(22) PCT Filed: Jan. 3, 2003
(86) PCT No.: PCT/IB03/00022

§ 371 Date: Aug. 14, 2006

(51) Int. Cl. C07C 63/04

(52) U.S. Cl. 562/401; 562/493

(57) ABSTRACT

The present invention relates to an environmentally benign process for preparation of (+)2-(4-chlorophenyl)-3-methyl butanoic acid (+ CPA) from its racemic acid, using optically active arylamines like (−) PEA in hydrophilic/hydrophobic organic solvents like butanol, propanol etc. as aqueous mixtures, separating the desired (+) CPA salt, mother liquor by filtration and refining the (+) CPA salt in the same solvent system as used for resolution, recovering the desired acid in high optical purity by extracting with aqueous mineral acid. The mother liquor is concentrated under vacuum and extracted with aqueous mineral acid to obtain undesired (−) CPA which was recovered and recycled after racemization. The aqueous mineral acid layer thus obtained is mixed with corresponding aqueous mineral acid layer obtained from (+) CPA recovery and extracted with aqueous caustic lie solution to recover the optically active amine used for resolution. Thus the method described effectively provides a process for recovery and recycle of the undesired (−) CPA, optically active amine, besides obtaining the desired (+) CPA in high optical purity.

### FIELD OF THE INVENTION

The present invention relates to an environmentally benign process for preparation of (+)-2-(4-chlorophenyl)-3-methyl butanoic acid. More particularly, the present invention provides a method for optical resolution of (±)-2-(4-chlorophenyl)-3-methyl butanoic acid (hereinafter referred to as CPA) which may be carried out in water and partly/totally miscible organic solvents preferably alcohols containing C₃-C₅ carbon chain. The method provides a simpler process besides the effective recovery and recycle of undesired isomer (−)CPA, resolving agent, and the organic solvent employed thereby resulting quantitative yields. The method of invention makes it possible to use same solvent system for the process of refining the salt to obtain high optical purity of CPA in one refinement which makes the process simpler, less cumbersome, more efficient, and thereby advantageous for industrial application.

### BACKGROUND OF THE INVENTION

CPA is an important component, of commercially important synthetic pyrethroids such as fenvalarate, flucythrinate, esfenvalerate etc. The bioefficacy of esters (A alpha isomer of fenvalarate) obtained by reaction of optically active (+) CPA acid is increased by two to four folds in comparison to that of esters of racemic caboxylic acids.

Reference is made to UK Patent Application GB 2014137A, wherein the resolution of CPA using aqueous ethanol in large quantities is described. The drawback of this process is use of large quantity of aqueous ethanol, and 1:1 equivalent of resolving amine and longer reaction time which prohibits its industrial application. The recovery of ethanol from aqueous solution further complicates the process in separation of the valuable solvent and its recycle.

JP Patent 55-136245 by Sumitomo Chemical Company Ltd. Japan claims a method for the optical resolution of (+) CPA with optical purity of +45.93° of (+) CPA acid with an yield of 41.7% based on (±)CPA charged. However, this claims could not be reproduced in practice under the same experimental conditions as described therein. What was achieved was optical rotation of +40.5° against claimed value [(+)45.93°] and the claimed value could not be obtained even after five crystallizations and modifications in the experimental conditions (see Table 1 herein). This patent also employs three solvent system for resolution of (±)CPA and purification of salt is carried out in a different solvent system than used for resolution of acid. The inherent draw back of this cited reference is because of the use of different solvent systems for optical resolution causing the cross contamination of the solvents and the separation of which poses environmental and commercial problems and needs innovation to obviate these problems.

JP Patent 62-185044 describes the asymmetric reduction of olefin derivatives in presence of noble metal catalyst modified with optically active binaphthyl derivative under pressure. The draw back of this method, is poor recycle of expensive catalyst and very high pressure, both of which are difficult to adopt for commercial production.

Reference is made to EP 0 060 466A1 wherein the CPA is resolved using DEA. This method suffers from the disadvantage of repetitive crystallization to obtain (+) and (−) salts of CPA involving longer crystallization times (24 hrs) and also a different solvent system is employed for refinement of salt of CPA, which is disadvantageous for industrial application.

### OBJECTS OF THE INVENTION

The main object of the present invention is to provide a process for production of optically pure (+) CPA which obviates the drawbacks of the prior art detailed above.

It is another object of the invention to provide a process for the production of optically pure (+) CPA which with less number of refinements (one/two) and using the same solvent system used for resolution of (±)CPA for refinements.

It is another object of the invention to provide a process for the production of optically pure (+) CPA which enables the effective recovery and recycling of (−) CPA after racemization.

A further object of the invention is to provide a process for the production of optically pure (+) CPA wherein the recovery of the optically active resolving amine is done in an effective manner and its recycle is significant in conserving reagent and enhancing the cost effectiveness of the process.

A further object of the invention is to provide an environmentally friendly process for the production of optically active (+) CPA by enabling the recycling of the resolving agent, acid and the organic solvent used.

### SUMMARY OF THE INVENTION

Accordingly the present invention provides a process for the preparation of (+)-2-(4-chlorophenyl)-3-methyl butanoic acid which comprises reacting (±)-2-(4-chlorophenyl)-3-methyl butanoic acid (CPA) with a resolving agent comprising an amine in a hydrophobic/hydrophilic organic solvent in the presence of water, separating the desired amine salt and refining the salt with the same solvent system used for resolution and recovering the desired (+)CPA as well as undesired (−)CPA and amine resolving agent.

In one embodiment of the invention, the resolution is conducted by treating racemic CPA with an amine to precipitate a salt of one enantiomer of CPA.

In another embodiment of the invention, the solvent is selected from the group consisting of an aliphatic, cycloaliphatic, aromatic hydrocarbon, hydroxylic solvent and any mixture thereof.

In another embodiment of the invention, the solvent is selected from the group consisting of methanol, ethanol, propanol, isopropanol, butanol, iso-butanol, tert-butanol, toluene and any mixture thereof.

In another embodiment of the invention, the solvent is selected from the group consisting of butanol, propanol, water and any mixture thereof.

In another embodiment of the invention, the amine resolving agent is an optically active amine.

In a further embodiment of the invention, the optically active amine is an arylamine containing 8 to 20 carbon atoms.

A yet another embodiment of the invention, the arylamine is selected from the group consisting of α-phenyl-β-(p-tolyl)ethylamine, α-phenyl-β-phenylethylamine, α-phenylethylamine and N,N-dialkyl α-phenylethylamine.

In another embodiment of the invention, the arylamine is selected from the group consisting of N,N dimethyl, N,N diethyl, N,N dipropyl, N,N diisopropyl, N-methyl, N-ethyl and higher alkyl amines.

In yet another embodiment of the invention, the aryl amine is (S)(−)α-phenylethylamine.

In yet another embodiment of the invention, the amine resolving agent is used in amount of 0.4 to 0.65 mole per mole of (±)CPA.

In a flier embodiment of the invention, the amine is added in neat form or in the form of solution.

In a further embodiment of the invention, the amine is added in one lot or over a period of time ranging from 10-60 minutes.

In yet another embodiment of the invention, the amine is added at a temperature in the range of 30 to 100° C.

In another embodiment of the invention, the amine is added to the racemic CPA solution.

In another embodiment of the invention, the racemic CPA solution is added to the amine.

In another embodiment of the invention, the solvent used is in the range of 20-40% as aqueous solution and two to three times by weight based upon the amount of CPA used.

In another embodiment of the invention, the resolution reaction is carried out over a period of 2 to 6 hours.

In another embodiment of the invention, the amine salt formed is substantially in the form of a precipitate.

In yet another embodiment of the invention, the temperature range during separation of optically active salt is in the range of ambient temperature to 80° C.

In yet another embodiment of the invention, the crystallized salt is separated by filtration or centrifugation.

In another embodiment of the invention, the optically active amine salt obtained is refined in a hydrophilic solvent selected from the group consisting of methanol ethanol, propanol, isopropanol, butanol, 2-butanol, tert butanol and an aqueous mixture thereof.

In a further embodiment of the invention, the hydrophilic solvent is selected from the group consisting of butanol, propanol and an aqueous mixture thereof.

In another embodiment of the invention, the optically active salt is refined at a temperature ranging from 40 to 120° C.

In another embodiment of the invention, the solvent used for refinement is in the range of 20-40% as aqueous solution and one to four times by weight based on the amount of optically active salt used.

In a further embodiment of the invention, the duration of refinement is in the range of 3-5 hrs.

In yet another embodiment of the invention, the optically active salt is separated after refinement at a temperature in the range of 40 to 70° C.

In a further embodiment of the invention, the optically active salt after refinement is separated by filtration or centrifugation.

In another embodiment of the invention, the optically active salt of (+) CPA after refinement is liberated using mineral/organic acids.

In another embodiment of the invention, the mineral acid used for liberation of optically active acid is selected from hydrochloric acid and sulphuric acid, preferably aqueous sulphuric acid.

In another embodiment of the invention, the aqueous mineral acid layer containing amine salt is combined with aqueous mineral acid layer obtained from recovery of the undesired (−) CPA.

In another embodiment of the invention, the mother liquor enriched with undesired (−) CPA salt obtained after precipitating the desired (+) CPA salt is concentrated at reduced pressure for recovery of (−) CPA.

In another embodiment of the invention, the undesired (−) CPA salt after concentration is treated with aqueous mineral/organic acids and extracted with hydrophilic/hydrophobic organic solvents and concentrated under reduced pressure for obtaining (−) CPA.

In another embodiment of the invention, the mineral acid used for liberation of (−) CPA from its amine salt is selected from hydrochloric acid and sulfuric acid, more preferably aqueous sulfuric acid.

In another embodiment of the invention, the liberated acid is treated with organic solvents like Dichloromethane, Dichloroethane, Chloroform, Toluene, Hexane, preferably Toluene.

In another embodiment of the invention, the aqueous mineral acid layer containing amine salt is combined with the corresponding aqueous mineral acid layer obtained from the liberation of desired (+) CPA to effect the recovery of optically active resolving agent.

In another embodiment of the invention, the aqueous mineral acid layers obtained from liberation of (+) CPA and (−) CPA are mixed, cooled preferably to 10 to 5° C. and extracted with aqueous caustic lye solution of concentration ranging from 20-80%, more preferably 30-60% to recover the resolving amine employed in resolution of (±) CPA.

In another embodiment of the invention, the crude amine obtained is used in subsequent batches of (±) CPA and the alkaline layer is extracted with hydrophilic/hydrophobic organic solvents such as benzene, toluene, hexane, dichloromethane, dichloroethane, Chloroform preferably benzene, toluene and hexane, preferably toluene.

### DETAILED DESCRIPTION OF THE INVENTION

The process of the invention involves recovery and recycling of the undesired R-isomer, recover and recycle of the expensive resolving agent and use of a solvent system wherein the loss of solvent to environment is minimized. In this quest for development of a suitable methodology, a comprehensive search for a better solvent system was undertaken to overcome the problems encountered in referred patent JP-55-136245. The results are tabulated in Table-2 (page Nos. 28 to 34). After evaluation of the data of Table-2 it was observed that optical resolution can be carried out in water and partly/totally miscible organic solvents preferably alcohols like butanol, propanol for the preparation of (+) CPA.

The present method is carried out in water and partly/totally miscible organic solvents and the latter being preferably, alcohols containing C₃-C₅ carbon units and to use the same solvent system for refining process of diastereomeric salt so as to obtain +CPA with high optical purity. Despite many methods known in literature, the chemical method of resolution through salt formation with organic base and its fractional crystallization or the diastereomeric salt formation with optically active amine, is preferred and the latter being more practicable for industrial application

In an embodiment of the present invention, the optical resolution of (+) CPA is performed by its diastereomeric salt formation using a resolving agent. The result of resolution is determined by two equilibria as disclosed in the article “A convenient method for optical resolution via diastereomeric salt formation” by M.ACS et al in Tetrahedron Vol. 41, No. 12, pp 2465-2470”. The theoretical possibility for a resolution via diastereomeric salt formation is due to differences in physico chemical properties of the diastereomeric salt pairs formed during the course of reaction of racemate (AB) and resolving agent (RH) with opposite chemical character.

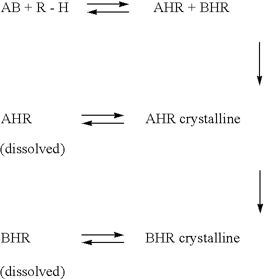

The virtual (chemical) yield is dependant on solubility of the salt in a given solvent system but optical yield is controlled by solubility differences. The solubility differences of salts is effected by a chiral and achiral factors. The resolutions mediated by diastereomeric salts depend principally on solubility differences and on the equilibration between salt and solution. It is important to note that sometimes the insoluble diastereomeric salt crystallizes out and the more soluble distereomeric salt is likely to undergo exchange with the racemate thereby increasing the formation of more insoluble diastereomeric salt provided the resolving agent used is less than stoichiometric amount. This phenomenon will yield preferentially diastereomeric salt of one of the enantiomers more than the other. During the course of resolution in a two solvent system the reactants are soluted in their convenient solvent. The resolving agent solution is added to that of racemate with stirring. In this two phase system, a rather complicated equilibrium takes place at the liquid-liquid interface as a result of which the crystallization/precipitation starts and continues at liquid-liquid interface which necessitates continuous mixing. The crystallization/precipitation is carried out by cooling at a predetermined temperature for a certain period of time.

The optical resolution is carried out using commercially available optically active amines such as different acyclic, heterocyclic, aromatic amines, basic amines like, brucine, cinchonine, morphine, stychine, basic aminoacids, glycine, agrinine, and the like, more preferably optically active amine is an aryl amine containing 8 to 20 carbons especially, alpha-phenylethyl amine (herein after referred as PEA) or α-phenyl-β(p-tolyl)ethylamine, α-phenyl-β-phenylethyl amine or N,N-dialkyl α-phenylethylamine or N,N-dimethyl or di-propyl, di-isopropyl or N-methyl, N-ethyl or higher alkyl amines.

The optical resolution of (±)CPA as for the solvent used in the reaction, the main criteria is the crystallization/precipitation of the desired diastereomeric salt from the solvent system employed usually water, hydroxylic or an aliphatic, aromatic or carbocyclic hydrocarbon solvents like alcohol having 1-5 carbon atoms such as methanol, ethanol, n-propanol, n-butanol, isobutanol, sec.butanol, tert-butanol, ethylene glycol, methoxy ethanol isopropylalcohol, or preferably alcohol containing 3-4 carbon atoms such as n-propanol, isopropanol, or preferably alcohol containing, n-butanol, tert. Butanol, sec.butanol, more preferably butanol, propanol, water or mixtures thereof. In the present invention the said alcohols are mixed with water in different proportions form 0-100% more preferably in the range of 20-40%.

The quantity of resolving agent used for the optical resolution of racemic CPA is variable and it is in the range of 0.5-1.0 mole against one mole of acid, more preferably in the range of 0.4-0.8. Resolving agent can be added in one lot or over a period of time ranging from 5 minutes to 120 minutes, more preferably 10-40 minutes at a temp. from 25° C. to 100° C. more preferably from 30° C. to 70° C.

The (±)CPA is reacted with optically active amine in the solvent as mentioned above in the presence of appropriate amount of water. The temp. of reaction is not limited, but it is desirable to keep temp. at 40°-150° during or after the reaction, in order to obtain (+) CPA of high optical purity while keeping the temp. at above mentioned range, the precipitated salt of (+) CPA and amine, is separated from mother liquor preferably by slow cooling. At this stage, the remaining CPA in mother liquor is in (−) form. The separating temp. is 25° to 70° C. or more preferably 30° to 55° C. The separation of PEA salt is effected by filtration/centrifugation while cooling the filtrate by external means/or at ambient temp. The (+)CPA-(−)PEA salt is washed with the same solvent system used for resolution to remove any adhering mother liquor and separated by filtration/centrifugation. The washings obtained is kept aside for use in washings of salt of subsequent batches or mixed as the case may be. The (+)CPA-(−) PEA salt thus obtained is subjected to refinement either by using fresh solvent system as used in resolution step or by the filtrate obtained from previous batches of refinement provided the optical rotation is in the acceptable range. The slurry thus obtained is refluxed for a period ranging from 60 to 240 minutes more preferably 60-180 mts. and then cooled to 35°-70° C. more preferably 45°-60° C. over a period of 60-360 minutes more preferably 60-180 minutes followed by filtration/centrifugation at above mentioned temp. The (+)CPA-(−) PEA salt obtained if necessary is recharged into reactor and alcoholic aqueous Solvents more preferably butanol water in the concentration of 20-40% water, more preferably 30-35% is added and heated to reflux for about 20-120 minutes more preferably 30-40 minutes, followed by filtration/centrifugation. The (+)CPA-(−) PEA salt is dried in vacuum oven at a temperature ranging from 50°-80° C. preferably at 50-60° C. The filtrate is recycled for refinement of (+)CPA-(−) PEA salt of fresh batches as such without any further operations, as mentioned above. The dried (+)CPA-(−) PEA salt thus obtained is liberated with mineral or organic acids like hydrochloric acid, sulfuric acid, acetic acid in aqueous medium. The liberated (+)CPA is extracted into organic solvent like chloroform, Dichloromethane, Dichloroethane or mixtures thereof, or aromatic hydrocarbons like toluene, benzene and the organic layer is concentrated under vacuum at 50°-80° C. to obtain (+)CPA. The acidified aqueous layer obtained after recovery of (+)CPA is kept aside to recover the resolving amine.

The mother liquor contain the undesired optically active CPA moiety (− form) usually as amine salt and washings of (+)CPA-(−)PEA salt obtained from resolution, is treated to recover the undesired acid. Normally the mother liquor is subjected to distillation under reduced pressure of 20-100 mm of Hg, more preferably 25-30 mm of Hg. at a still temp. from 40°-100° C., more preferably 60°-80° C. The distillate obtained is recycled after estimation of moisture content. The bottoms, thus obtained is made free of organic solvent and acidified with mineral acids such as hydrochloric acid, sulfuric acid, organic acids like acetic acid or aqueous Alkali such as sodium or potassium hydroxide or calcium hydroxide for recovery of (−) CPA or resolving agent as the case may be.

The (−)CPA-(−) PEA salt is acidified with mineral or organic acids like hydrochloric acid, sulfuric acid, acetic acid in aqueous medium. The liberated (−)CPA is extracted into organic solvent like chloroform, Dichloromethane, Dichloroethane or mixtures thereof, or aromatic hydrocarbons like toluene, benzene and the organic layer is concentrated under vacuum at 50°-80° C. to obtain (−)CPA which is racemized for further use in resolution. The acidified aqueous layer containing (−) PEA is taken for recovery of optically active resolving agent by combining with acidified aqueous layer obtained from (+) CPA recovery.

The two acidified aqueous layers obtained after recovery (+)CPA and (−) CPA are mixed as aqueous layer streams and cooled to 5-20° C., more preferably 5-10° and extracted with aqueous alkali solution of concentration ranging from 10-80% more preferably 30-60% resulting in separation of crude layer of optically active resolving agent which is separated out. Aqueous alkali solution is extracted with aromatic hydrocarbon solvents like benzene, toluene or chlorinated hydrocarbon solvents like chloroform, Dichloromethane, Dichloroethane and concentrated to recover optically active resolving agent. Optically active resolving agent thus obtained is recycled for further batches after ascertaining optical purity.

The following examples are given by way of illustration and therefore should not be construed to limit the scope of the present invention.

### EXAMPLE—1

In a suitable reaction vessel, 42.2 g of (±)-CPA and 138.0 g of 30% aqueous n-propanol was charged and heated to form a solution. A solution of 14.2 g (−) PEA in 42 g of 30% aqueous n-propanol was added to the above solution at 52° C. The mixture was heated to the reflux temperature for (88° C.) about 60 minutes and the contents were allowed to reach to 37° C. under stirring in about 120 minutes. The precipitated (+) CPA-(−) PEA salt (cake) was filtered off and washed with 50 g of 30% aqueous n-propanol twice separating the filtrate each time. The wet cake was dried and weighed. The general procedure followed for liberation of the (+)CPA from its amine salt was described below. A small portion of the salt (2.0 g) was extracted with 30 ml of 40% Sulphuric acid. The liberated (+) CPA, was extracted with 2×20 ml of DCM and concentrated to obtain (+) CPA, which was dried and analysed for its optical purity by polorimetry. The same procedure was followed for all the examples described below. Dry weight=23.16 g. α_{D}=+41.09° [CHCl₃, C=6.00]

### EXAMPLE—2

To 42.26 g of (±)-CPA was added under stirring 120 g of 20% aqueous n-butanol to form a solution. A solution of 14.26 g of (−) PEA in 40 g of 20% aqueous n-butanol was added to above solution at 55° C. The reaction mixture was refluxed for about 90 minutes and allowed to cool slowly to 37° C. in about 90-120 minutes. The precipitated CPA-(−)PEA salt (cake) was filtered off and washed with 50 g of 20% aqueous butanol under stirring twice separating the filtrate each time. The wet cake was dried and weighed. Dry weight=23.5 g. α_{D}=+41.14 [CHCl₃; C=6.23]

### EXAMPLE—3

To 42.2 g of (±) CPA was added under stirring 94.0 g of 20% aqueous n-propanol to make a solution. A solution of 14.2 g of (−) PEA in 71.0 g of propanol-water (20%) was added to the above solution at 50° C. and the mixture was heated to reflux temperature for about 60-70 minutes, allowed to cool to 30° C. under stirring in about 120 minutes. Precipitated salt was filtered and cake was washed with 20% aqueous n-propanol twice, separating the filtrate each time. Cake obtained was dried and weighed. Dry weight=23.44 g. α_{D}=+40.56 [CHCl₃; C=6.05]

### EXAMPLE—4

To 23.44 g of above PEA salt, 72.3 g of 20% aqueous propanol was added under stirring and the contents were heated to reflux for about 120-130 minutes. The reaction mixture was allowed to cool to 37° C. and the precipitated salt was filtered off and washed with 50 g of the above solvent system. The cake obtained was dried after separating filtrate. Dry weight=21.2 g. α_{D}=+43.78 [CHCl₃; C=6.06]

### EXAMPLE—5

To 42.2 g of ± CPA was added under stirring 45.0 g of saturated solution of butanol in water to make a homogeneous solution. A solution of 14.4 g (−) PEA in 114 g of saturated solution of butanol in water was added to the above solution at a temperature ranging between 45-52° C. and the mixture was heated to reflux temperature for about 65 minutes, allowed to cool to 37° C. in about 150 minutes under stirring. Precipitated salt was filtered. Cake was washed with 50 g of 10% aqueous butanol twice, separating the filtrate each time. Cake obtained was dried and weighed. Dry Weight=26.71 g. α_{D}=+40.79 [CHCl₃; C=6.03]

### EXAMPLE—6

To 42.2 g of (+)-CPA was added under stirring, 80.0 g of 20% aqueous isobutyl alcohol to form a solution. A solution of 14.2 g (−) PEA in 78 g of 20% aqueous isobutyl alcohol was added to above solution at 60° C. The reaction mixture was heated to reflux temperature for about 65 minutes and the contents were allowed to reach to 37° C. under stirring in about 120 minutes. The precipitated salt was filtered and cake was washed with 50 g of 20% aqueous isobutylalcohol twice. The cake obtained is dried and weighed. Dry weight: 25.60 g. α_{D}=+40.78 [CHCl₃; C=6.06]

### EXAMPLE—7

To 42.1 g of (±)CPA was added under stirring, 155 g of 21% aqueous n-butanol-n-propanol to form a homogeneous solution. A solution of 14.2 g of (−) PEA in 105 g of 21% aqueous n-butanol-n-propanol was added to the above solution at 70° C. in about 50 minutes and the mixture was heated to reflux for about an hour, allowed to reach to room temperature under stirring in about 90-120 minutes. Crystallized (+) CPA(−) PEA salt was filtered off and cake was washed twice with 50 g of same solvent system used for reaction separating the filtrate each time. Wet cake was dried and weighed. Dry weight=21.17 g. α_{D}=+41.570 [CHCl₃; C=6.06]

### EXAMPLE—8

To 42.8 g of (±)CPA was added under stirring 186.0 g of 30% aqueous n-propanol to form a solution. A solution of 14.2 g of (−) PEA in 71.0 g of 30% aqueous n-propanol was added to the above solution at 55° C. in 30 minutes. The reaction mixture was heated to reflux for about 150 minutes and the contents were allowed to reach to room temperature. The precipitated salt of (+) CPA-(−) PEA was filtered off. The cake was re-dissolved in 50 g of the solvent system used for resolution of (+) CPA, heated to reflux for about 30 minutes cooled and filtered off The same procedure was repeated again and the cake obtained was dried and weighed. Dry Weight=21.01 g. α_{D}=+42.86 [CHCl₃; C=6.04]

### EXAMPLE—9

To 42.2 g of (±)CPA was added under stirring, 85 g of 3.5% aqueous toluene to form a solution. A solution of 14.2 g (−) PEA in 56 g of 3.5% aqueous toluene was added to above solution at 40° C. in 20-30 minutes and contents were heated to reflux for about 200 minutes and cooled to room temperature. Precipitated salt was filtered and cake was washed with 75 g of toluene twice separating the filtrate each time. Wet cake was dried to a constant weight. Dry weight=34.00 g. [α]_{D}=+34.528 [CHCl₃; C=6.02]

### EXAMPLE—10

To 42.2 g of (±) CPA having [α]_{D}-3 was added under stirring, 138 g 30% aqueous n-butanol to form a solution. A solution of 14.2 of (−) PEA in 42.2 g of above solvent system was added to the already made (±)-CPA solution at 50°-60° C. in 30-50 minutes. The reaction mixture was refluxed for about 60-80 minutes, cooled to room temperature and filtered off. Precipitated salt (cake) was washed twice with 50 g of the solvent system used for reaction, separating the filtrate each time. Wet cake was dried and weighed. Dry weight=23.15 g. [α]_{D}=+38.11 [CHCl₃; C=6.05]

### EXAMPLE—11

In a suitable reactor 566 g of (±) CPA and 2430 g of 30% aqueous butanol was charged under stirring. A solution of 322 g of (−) PEA in 966 g of 30% aqueous n-butanol was added to above solution at temperature ranging from 50°-70° C. in about 60 minutes and the contents were heated to reflux for about 90 minutes. The reaction mixture was allowed to come to about 50° C. in about 240 minutes and filtered off. The filtrate was weighed (2507 g) and treated for recovery of R enriched (−) CPA isomer along with washings of wet cake (850 g) as described in example (11A). The cake (757 g) obtained was washed twice (2×390 g) with 30% aq. n-butanol and filtered off. The cake (680.0 g) was further recrystallised using 2430 g of 30% aqueous n-butanol by heating to reflux temperature (≈90° C.) for about 120 minutes and then allowed to cool to 52° C. in about 150 minutes and filtered off. The weight of cake and filtrate being 527.7 g and 2523 g respectively. The wet cake (527.7 g) was refined in 1562 g of 30% aqueous n-butanol by refluxing for 35 minutes at 90° C. and the contents were cooled to 52° C. in about 60 minutes, filtered and weighed. The weight of cake and filtrate obtained was 406.3 g and 1617 g respectively. The cake was dried to a constant weight (306 g). The dried PEA salt (306 g), distilled water (300 g), toluene (900 g) were charged into a suitable reactor successively and contents were stirred well. 250 g of 40% H₂SO₄ was added over a period of 10-30 minutes and mixed well for 20-30 minutes. The separated aqueous layer containing PEA-HSO₄ (641 g) was stored for recovery of S (−) PEA. The toluene layer is made free of traces of acid and concentrated to obtain (S+) CPA (197 g). Dry weight of S (+)CPA=197.0 g. [α]_{D}=+45.129 [CHCl₃, 6.01]

### EXAMPLE—11 A

The filtrate (2507 g) and washings of wet cake (850 g) obtained from above example of resolution of (±) CPA was concentrated under reduced pressure to remove solvent (butanol) at a temperature ranging from 60-78° C. after which 200 g of 40% Sulphuric acid was added to the residue and mixed thoroughly for 30-40 minutes at that stage, toluene (500 g) was added and stirred further to extract the liberated (−)CPA acid into the toluene layer. The contents were transferred into a separator and the lower acidified aq. layer containing PEA-HSO₄ (RA-1; 407 g) was separated and mixed with acidified aqueous layer obtained from (+) CPA recovery for liberating the resolving amine, as described in example (11 B). The toluene layer (733 g) was made free of traces of acid by washing with distilled water and concentrated under reduced pressure to obtain (−) CPA (320 g) which was subsequently racemised.

### EXAMPLE—11 B

The acidified aqueous layers (SA-1, 641 g; RA-1, 407 g) obtained after recovery of (+)CPA and (−)CPA respectively are mixed in a suitable reactor and cooled to 0°-5° C. under constant stirring. 200 g of 50% caustic lie was added over a period of 10-15 minutes and the layers were allowed to separate. The upper layer containing (204 g) of crude PEA was separated and aqueous layer was extracted with toluene twice (2×100 g) and the toluene layers were analysed for PEA content to be used for further batches of resolution of (±)CPA.

### EXAMPLE—12

The glass lined reactor equipped with stirrer, heater exchanger, dropping funnel and a thermovel is charged with 6.36 kg of (±) CPA, 19.17 kg butanol, 8.79 kg water under stirring and heated to 50-60° C. 2.27 kg of S (−) Phenylethylamine (PEA) is fed into the reactor over a period of 20-30 minutes. The contents are heated with steam to vigorous reflux (92-93) by circulating cold water in the heat exchanger and reflux is contd. for 60 minutes, followed by gradual cooling of the reaction mixture to 35° C. over a period of 120 minutes and is filtered off (28.32 kg ML-I). The (+) CPA-(−) PEA salt (cake) obtained is dried under vacuum for a period of 20-30 minutes. The cake is recharged into the reactor followed by of 6.38 kg of butanol, 2.78 kg of water and contents are stirred for a period of 20 minutes, filtered under vacuum to dryness to obtain 10.02 kg of washings (WS-I) and 6.38 kg of cake. The filtrate (ML-1) and washings (WS-1) are combined and concentrated for recovery of R enriched (−) CPA. The cake (6.38 kg) obtained is charged into the reactor and recrystallized using 19.18 kg of Butanol and 8.3 kg of water by heating the reaction mixture to vigorous reflux under stirring (92-93° C.), maintained at that temperature for 60-75 minutes and then allowed to cool to 55-60° C. over a period of 120 minutes. Recrystallized slurry is filtered under vacuum (120-100 mm). The filtrate obtained (27.06 kg) is kept in storage tank to be used for subsequent batches of recrystallization of cake. The cake obtained (5.26 kg) is further refined by charging into the reactor using 4.47 kg butanol, 1.93 kg of water and heating to reflux (92-93° C.) for 30-40 minutes and then cooled to 45° C. over a period ranging from 45-60 minutes under stirring and the slurry is filtered after removing most of the solvent the cake is dried under vacuum (120-100 mm) for 10-15 minutes. The filtrate (5.3 kg) is stored and recycled to be used for refinement of further batches of the salt. The cake (3.98 kg) is dried in a jacked vacuum tray drier at 46-48° C. under reduced pressure till a constant wt. (3.08 kg) is obtained.

The salt of (+) CPA-(−) PEA (3.08 kg) thus obtained is charged into a 20 lit glass stirred reactor, followed by 2.96 kg of distilled water and 9.1 kg of toluene. 2.37 kg of 40% aqueous Sulphuric acid is added to the contents of the reactor over a period of 30-40 minutes under stirring and mixed well for 15-20 minutes, after which the layers are allowed to separate. The acidified aqueous layer (SA-1; 6.41 kg) containing PEA-HSO₄ is stored for recovery of (−) PEA The toluene layer is, washed with distilled water (3×3.0 kg) till pH. of aqueous layer is neutral The aqueous layers are kept aside for reuse in subsequent batches. The toluene layer is concentrated under reduced pressured (40-30 mm) at 40-50° C. to obtain 1.99 kg of (+) CPA of optical purity 42.8 (CHCl₃, C-6.05) which is subsequently converted to acid chloride to be used in preparation of Esfenvalerate.

The mother liquor (ML-1, 28.319 kg) and washing (WS-1; 10.02) obtained from above described resolution process of (±) CPA containing R enriched (−) CPA is fed into a Rotary evaporator equipped with vacuum system, and made free of butanol and water at a temperature of 60°-70° C. under vacuum (21-6 mm) by addition of 5.1 kg of fresh distilled water during distillation. After ensuring the complete removal of solvent, 1.97 kg of 40% aqueous Sulphuric acid is fed into the reactor and mixed for 20-40 minutes, followed by addition of 5.08 kg of toluene. The liberated (−)CPA-PEA sulfate solution is transferred into a 20.0 lit. glass stirred reactor and stirred for 20-30 minutes after which the layers were allowed to separate. The acidic aqueous layer containing (−)PEA—HSO₄ (RA-1; 4.2 kg) is stored to be mixed with corresponding acidic aq. layer obtained from (+)CPA acid recovery to liberate (−)PEA. Toluene layer is washed with distilled water (3×3.0 kg) till pH of aqueous layer is neutral. These washings are stored for reuse in subsequent batches. Toluene layer (8.39 kg) is concentrated in Rota evaporator, under vacuum (40-6 mm) at temperature of 40-50° C. to obtain 3.67 kg (−) CPA which is dried in vacuum tray drier at 40-45° C. to yield 3.3 kg of (−) CPA which is subsequently racemised to (±)CPA. The acidified aqueous layers (SA-1, 6.4 kg RA-1, 4.2 kg) obtained from recovery of corresponding (+)CPA and (−) CPA are mixed and charged into 20.0 lit. glass stirred reactor and cooled to 10-5° C. 2.04 kg of 50% caustic lye solution is added while stirring the contents in 60-75 minutes and the layers are allowed to separate out. Upper layer containing (−) PEA is separated out (2.14 kg) and stored to be used for further batches of racemization of (±)CPA. Aqueous layer is washed twice (2×2.0 kg) with toluene. Toluene layer was estimated for PEA content and recycled for further batches.

The main advantages of the present invention are:

1. The present invention makes it possible to obtain high optically pure (+)CPA with less number of refinements (one/two) and utilizes same solvent system as used for resolution of (±)CPA for refinements.
2. The another advantage of the invention is the effective recovery of the (−) CPA to recycle it after racemization.
3. The recovery of the optically active resolving amine in an effective manner and its recycle is significant in conserving reagent and enhancing the cost effectiveness of the process from economical considerations.

4. The process is comprehensive in that resolving agent, acid and the organic solvent used are effectively recovered and recycled thereby reducing the environmental burden.

TABLE-1

| | | | | Resolving | Reflux | Dry Wt. | Optical | |
|---|---|---|---|---|---|---|---|---|
| S. | | | Sol. System Toluene + | Amine (−) | Time | of salt | Rotation | |
| No. | RS Acid (gms) | | MeOH + H₂O (gms) | PEA (in gms) | (Hrs.) | (gms) | of (+) CPA | Remarks |
| 1. | 1063 | | 1068 + 837 + 225 | 366.0 | 2.15 | 803.0 | +37.45 | Pt 55-136245 |
| 2 | 803.0 | (Refinement) | 800 + 640 + 164 | — | 3.30 | 691 | +40.401 | |
| 3 | 692 | (Refinement) | 692 + 552 + 140 | — | 2.0 | 646 | +42.1 | |
| 4 | 660 | | 663 + 523 + 140 | 227 | 2.0 | 650.35 | +36.56 | Cooled to 40° C. in 3½ hrs filtered |
| | | | | | | | | washed with 80% MeOH twice |
| 5 | 650 | (Refinement) | 650 + 518 + 133 | — | 2.3 | 610 | +40.703 | Cooled to 48° C. in 1½ hr |
| 6 | 212.0 | | 212.5 + 170.9 + 42.9 | 75.72 | 2.0 | 143 | +37.7 | Cooled to 38° C. in 3 hrs |
| 7 | 143.0 | | 145 + 115 + 29 | — | 2.15 | 1.35 | +40.95 | Cooled to 30° C. in 1 hr. |
| 8 | 212.6 | | 212.7 + 170.80 + 42.8 | 72.77 | 2.0 | 147 | +40.07 | Cooled to 22° C. 3.20 hrs |
| 9 | 147.0 | | 160 + 128 + 32 | — | 2.15 | 135.82 | +40.5 | Cooled to 20° C. in 2.5 hrs |
| 10 | 1068.0 | | 1068 + 835 + 234 | 354.17 | 2.0 | 802.1 | +39.86 | Cooled to 31° C. in 2 hrs filtered |
| 11 | 802.0 | (Refinement) | 802 + 626 + 176 | — | 3.0 | 640 | +42.90 | Cooled to 33° C. in 1.15 hrs |
| 12 | 640.0 | (R-II) | — 1026 + 254 | — | 3.20 | 685 | +43.20 | Cooled to 34° in 1.0 hrs |
| 13 | 683.0 | (R-III) | — +1130 + 280 | — | 3.00 | 694 | +43.67 | Cooled 36° in 1 hrs |
| 14 | 694 | (R-IV) | — 1115 + 285 | — | 1.30 | 70.22 | +43.09 | Cooled to 36° C. in 45 minutes |
| 15 | 702.0 | (R-V) | 702 + 562 + 140 | | 2.15 | 669 | +43.3 | Cooled to 37° C. in 1 hrs 30 minutes |

| | | | | | | | |
|---|---|---|---|---|---|---|---|
| 16 | 53 | 53.4 + 43.0 + 10.6 | 18.6 | 2.10 | 35.5 | +31.0 | Maintained at 60° for 1 hr and filtered |
| 17 | 42.7 | 43.1 + 34.4 + 8.6 | 15.0 | 2.0 | 27.4 | +32.0 | Maintained at 55-60°, 1 hr, filtered |
| 18 | 42.1 | 42.2 + 34.5 + 7.60 | 15.05 | 2.0 | 31.92 | +28.0 | Maintained at 49-53°, 1 hr, filtered |
| 19 | 42.15 | 42.2 + 35.3 + 7.7 | 15.2 | 2.0 | 32.25 | +27.5 | Maintained at 46-43°, 1.15 hrs, filtered |
| 20 | 42.10 | 43.0 + 34.9 + 7.6 | 15.6 | 2.3 | 32.78 | +28.0 | Maintained at 40-36°, 1.15 hrs, filtered |
| 21 | 42.34 | 42.4 + 34.9 + 7.4 | 15.34 | 2.3 | 32.77 | +27.0 | Maintained at 60°, 1.40 hrs, filtered, |
| | | | | | | | cake washed with 80% methanol twice |
| 22 | 42.34 | 42.4 + 34.6 + 7.6 | 15.10 | 2.1 | 26.59 | +38.38 | Maintained at 31°, in 2 hrs, filtered, |
| | | | | | | | cake washed with 80% methanol |

TABLE-2

| | | Resolving | | Reflux | Filtration | Wt. of | Optical | |
|---|---|---|---|---|---|---|---|---|
| S. | RA acid | Agent | | Temp. | Temp. | Salt | rotation | |
| No. | wt(gms) | wt. (gms) | Solvent System | (° C.) | (° C.) | (gms) | (+)CPA | Remarks |
| 1 | 42.45 | 14.5 | IPA | 81 | 59 | 31.50 | +31.98 | Cake washed twice with IPA; |
| | | | | | | | | 50 m [symbol] each time |
| 2 | 42.45 | 14.45 | IPA | 83 | 69 | 32.58 | +31.14 | Cake washed twice with IPA; |
| | | | | | | | | 50 ml each time |
| 3 | 42.5 | 14.5 | IPA | 85 | 37 | 32.77 | +32.29 | Cake washed twice with IPA; |
| | | | | | | | | 50 ml each time |
| 4 | 42.37 | 14.3 | 20% aq. IPA | 81 | 38 | 28.05 | +36.85 | Cake washed twice with IPA; |
| | | | | | | | | 50 ml each time |
| 5 | 42.2 | 14.5 | n-propanol | 97 | 41 | 29.77 | +36.16 | Cake washed twice with |
| | | | | | | | | isopropanol; 50 ml each time |
| 6 | 42.0 | 14.4 | n-butanol | 110 | 39 | 28.55 | +37.60 | Cake washed twice with |
| | | | | | | | | butanol; 50 ml each time |
| 7 | 42.2 | 14.4 | Acetonitrile | 80 | 36 | 33.81 | +29.76 | Cake washed twice with |
| | | | | | | | | acetonitrile; 50 ml each time |
| 8 | 42.2 | 14.4 | Dioxane | 96 | 40 | 32.27 | +32.81 | Cake washed twice with |
| | | | | | | | | dioxane(60 gms; 86 gms) |
| 9 | 42.2 | 14.4 | 20% aq. n- | 91 | 38 | 23.44 | +40.55 | Cake washed twice with |
| | | | propanol | | | | | n-propanol 50 ml each time |
| 10 | 42.2 | 14.4 | n-butanol water | 92 | 38 | 24.59 | +40.79 | Cake washed twice with 10% |
| | | | saturated | | | | | aq. butanol; 50 ml each time |
| 11 | 42.2 | 14.2 | Isobutyl alcohol | 101 | 37 | 29.07 | +37.33 | Cake washed twice with |
| | | | | | | | | isobutylalchool; 50 ml each time |
| 12 | 42.2 | 15.07 | Diisopropylether | 68 | 39 | 37.83 | +24.22 | Cake washed twice with |
| | | | | | | | | diisopropylether; (80 gms; 83 ms) |
| 13 | 42.2 | 14.2 | 30% aq. propanol | 88 | 37 | 23.16 | +41.09 | Cake washed twice with 30% |
| | | | | | | | | propranol; 50 ml each time |
| 14 | 42.2 | 14.2 | Tertiary butanol | 83 | 34 | 33.38 | +29.25 | Cake washed twice with |
| | | | | | | | | tertiary butanol; 50 ml each time |
| 15 | 42.26 | 14.26 | 20% aq. n- | 95 | 37 | 23.53 | +41.14 | Cake washed twice with 20% |
| | | | butanol | | | | | n-butanol; 50 ml each time |
| 16 | 42.26 | 14.23 | 20% aq. | 91 | 37 | 23.60 | +40.78 | Cake washed twice with 20% |
| | | | isobutanol | | | | | iso-butanol; 50 ml each time |
| 17 | 42.5 | 14.25 | Ethanol | 80 | 38 | 23.92 | +39.09 | Cake washed twice with |
| | | | | | | | | ethanol; 50 ml each time |
| 18 | 42.2 | 14.23 | Methanol | 68 | 37 | 23.20 | +39.99 | Cake washed twice with |
| | | | | | | | | methanol; 50 ml each time |
| 19 | 42.8 | 14.2 | 30% aq. n- | 89 | 38 | 20.28 | +41.42 | Cake washed twice with 30% |
| | | | propanol | | | | | n-propanol; 50 ml each time |
| 20 | 42.2 | 14.2 | 20% aq. diaxone | 90 | 37 | 20.97 | +37.16 | Cake washed twice with 20% |
| | | | | | | | | dixaone; 50 ml each time |
| 21 | 48.2 | — | 30% aq. n- | 89 | 37 | 41.25 | +43.78 | Cake washed twice with 30% |
| | | | propanol | | | | | n-propanol; 50 ml each time |
| 22 | 42.2 | 14.42 | 30% aq. methanol | 73 | 37 | 26.31 | +38.44 | Cake washed twice with 30% |
| | | | | | | | | methanoll; 50 ml each time |
| 23 | 42.24 | 14.2 | n-butanol water, | 93 | 37 | 23.03 | 39.99 | Cake washed twice with saturated |
| | | | methanol | | | | | n-butanol water; 50 ml each time |
| 24 | 41.25 | Recryst-2 | 30% aq. n- | 89 | 34 | 33.76 | +44.91 | Cake washed twice with 30% aq. |
| | | | propanol | | | | | n-propanol; 50 ml each time |
| 25 | 42.2 | 14.22 | n-butanol, 30% | 82 | 37 | 27.83 | +38.31 | Cake washed twice with 30% n- |
| | | | methanol | | | | | butanol, methanol; 50 ml each time |
| 26 | 33.76 | Recryst-3 | 30% aq. n- | 88 | 37 | 29.33 | +44.86 | Cake washed twice with 30% aq. |
| | | | propanol | | | | | n-propanol; 50 ml each time |
| 27 | 42.38 | 14.23 | 40% aq. n- | 89 | 37 | 23.93 | +40.54 | Cake washed twice with 40% |
| | | | propanol | | | | | n-propanol; 50 ml each time |
| 28 | 42.18 | 14.23 | 50% aq. n-propanol | 89 | 37 | 22.96 | +40.41 | Cake washed twice with 50% |
| | | | | | | | | n-propanol; 50 ml each time |
| 29 | 42.1 | 14.2 | n-propanol, | 90 | 37 | 21.17 | 41.57 | Cake washed twice with n-PA, n-BA, |
| | | | n-butanol, water | | | | | water; 50 ml each time |
| 30 | 42.8 | 14.2 | 30% aq. n- | 88 | 37 | 24.25 | 42.8 | Cake washed twice with 30% aq. n- |
| | | | propanol | | | | | PA, 50 ml each time, hot washings |
| 31 | 42.2 | 14.2 | 30% aq. n- | 92 | 37 | 26.21 | 41.47 | Cake washed twice with 30% aq. n- |
| | | | butanol | | | | | BA, 50 ml each time, hot washings |
| 32 | 42.2 | 14.2 | 30% aq. n- | 92 | 37 (10 | 25.47 | +42.56 | Two hot washings of cake by heating |
| | | | butanol | | minutes, | | | for 10 minutes to 92°, cooled to RT |
| | | | | | 92°) | | | filtered, 50 gms of wash each time |
| 33 | 42.2 | 14.2 | 30% aq. n- | 93 | 37 | 22 | +40.71 | Cake washed twice, dilution 1:6, |
| | | | butanol | | | | | cold washings |
| 34 | 42.23 | 14.25 | 30% aq. n- | 92 | 50° hot | 20.58 | +41.83 | Cake washed twice by heating to 50° |
| | | | butanol | | filtration | | | for 2 hrs. and filtered at 60°; |
| | | | | | | | | 50 ml each time |
| 35 | 42.23 | 14.2 | 40% aq. n- | 92 | 37 | 226.64 | +40.97 | Cake washed twice with 40% aq. |
| | | | butanol | | | | | n-butanol; 50 ml each time |
| 36 | 42.23 | 14.2 | 40% aq. n- | 93 | 50 | 24.24 | +41.08 | Cake washed twice with 40% aq. |
| | | | butanol | | | | | n-butanol by heating to 50° for |
| | | | | | | | | 2 hrs and filtered at 60° |
| 37 | 42.2 | 14.28 | Toluene, 4 ml | 90 | 37 | 34.00 | +34.52 | Cake washed twice with 75 gms |
| | | | water | | | | | toluene |
| 38 | 42.2 | 14.2 | 30% aq. n- | 92 | 37 | 23.15 | +38.11 | Cake washed twice with 50 |
| | (α-3) | | abutanol | | | | | gms of 30% aq. butanol |
| 39 | 42.27 | 14.26 | 30% aq. n- | 93 | 37 | 18.00 | +39.56 | Cake washed twice with 50 |
| | (α-1) | | abutanol | | | | | gms of 30% aq. butanol |
| 40 | 566 | 194 | 30% aq. n- | 93 | 42 | 318.53 | +42.10 | Cake refluxed for 10 mts. with 30% |
| | | | abutanol | | | | | butanol, cooled to 40°, filtered, |
| | | | | | | | | 50 ml used for washing cake with |
| | | | | | | | | solvent system. |
| 41 | 566 | 194 | Rec. n-butanol | 93 | 42, first | 345.40 | +41.40 | Cake refluxed for 10 mts. with 30% |
| | | | (21.9 Wt/wt) | | wash 44° | | | aq. butanol, cooled to 40°, |
| | | | | | | | | filtered, 5 [symbol] ml used for washing cake with |
| | | | | | | | | solven [symbol] system. |
| 42 | 663.15 | Recrys- | Rec. + fresh | 93 | 55, RT | 536.00 | +44.05 | Filtered at 55° and two cold |
| | | talization | 30% aq. butanol | | | | | washings with 50 ml aq. butanol |
| 43 | 55.7(PEA | — | 30% aq. butanol | 93 | 37, RT | 38.32 | +43.79 | Cake refluxed for 10 mts. with 30% aq. |
| | salt) + | | | | | | | butanol at 92°, cooled to RT, |
| | 24.5 RS | | | | | | | filtered, and process repeated again |
| | acid | | | | | | | |
| 44 | 55.7(PEA | — | 30% aq. butanol | 93 | 37 | 48.06 | +45.14 | Cake refluxed for 10 mts. with 30% aq. |
| | salt) + | | | | | | | butanol at 92°, cooled and filtered, |
| | 24.5 RS | | | | | | | and repeated the process again |
| | acid | | | | | | | |
| 45 | 55.7(PEA | — | 30% aq. butanol | 93 | 37 | 47.17 | +44.98 | Cake refluxed for 10 mts, with 30% aq. |
| | salt) + | | | | | | | butanol at 92°, cooled, filtered, |
| | 24.5 RS | | | | | | | process repeated again |
| | acid | | | | | | | |
| 46 | 42.24 | 19.24 | 30% aq. butanol | 93 | 37 | 31.05 | — | Cake refluxed for 10 mts. with 30% aq. |
| | | | | | | | | butanol at 92°, cooled and filtered |
| | | | | | | | | at 37° |
| 47 | 42.24 | 24.05 | 30% aq. n- | 91 | 37 | 33.06 | +38.48 | Cake refluxed for 10 mts. at 93° C. |
| | | | butanol | | | | | with 50 ml solvent system cooled to |
| | | | | | | | | 37° and filtered. |
| 48 | 29.05 | — | 30% aq. n- | 93 | 32 | 24.52 | +43.43 | Refluxed at 93° for 1 hr, cooled to |
| | (PEA | | butanol | | | | | 60° C., maintained at 60° for 3 |
| | salt) | | | | | | | hrs, filtered. Cake washed with 50 ml, |
| | | | | | | | | 30% Aq. butanol |
| 49 | 31.06 | — | 30% aq. n- | 91 | 52 | 25.71 | +44.43 | Refluxed at 91° for 1 hr, maintained |
| | (PEA | | butanol | | | | | at 55-52° for 2 hrs, and filtered |
| | salt) | | | | | | | |
| 50 | 42.24 | 24.05 | 30% aq. n- | 91 | 37/42 | 28.00 | +43.06 | Refluxed 1 hr at 91°, filtered at |
| | | | butanol | | | | | 42° C. Cake washed with 50 ml solvent |
| | | | | | | | | system and recrystallized at 91° by |
| | | | | | | | | reflux for 1 hr, maintained at 50° |
| | | | | | | | | for 1 hr, filtered, cake washed with |
| | | | | | | | | 50 ml solvent system |
| 51 | 1058.0 | — | 30% aq. n- | 91 | 37/52 | 866.0 | +45.38 | Recrystallised by reflux at 91° for 1 |
| | (PEA | | butanol | | | | | hr cooled to 55°, maintained at 55° |
| | salt) | | | | | | | for 2 hrs, filtered. Cake washed with |
| | | | | | | | | 1009 gm of solvent system. |
| 52 | 274 | — | 30% aq. n- | 93 | 55/57 | 167.0 | +44.20 | Recrystallised by reflux at 93° for 1 |
| | (PEA | | butanol | | | | | hr maintained at 55-57° for 1½ |
| | salt) | | | | | | | hrs, filtered, cake washed with 274 gms |
| | | | | | | | | solvent system |
| 53 | 566 | 322 | 30% aq. n- | 93 | 49 | 680.0 | +35.39 | Recrystallised by reflux at 91° for 1 |
| | | | butanol | | | | | hr, maintained at 55-50° for |
| | | | | | | | | 1½ hrs, cake washed with |
| | | | | | | | | 782 gms of aq. butanol |
| 54 | 680 | — | | 93 | 52/52 | 680.0 | +45.12 | Recrystallised by reflux at 92° for 1 |
| | (PEA | | | | | | | hr, maintained at 52-56° for 1 hrs, |
| | salt) | | | | | | | filtered, cake recrystallised at 92° |
| | | | | | | | | for 30 mts. Cooled to 52°, filtered. |

We claim:

1. A process for the preparation of (+)2-(4-chlorophenyl)-3-methyl butanoic acid which comprises reacting (±)2-(4-chlorophenyl)-3-methyl butanoic acid (CPA) with a resolving agent comprising an amine in a hydrophobic/hydrophilic organic solvent in the presence of water, separating the desired amine salt and refining the salt with the same solvent system used for resolution and recovering the desired (+)CPA and undesired (−)CPA and amine resolving agent.

2. A process as claimed in claim 1 wherein the resolution is conducted by treating racemic CPA with an amine to precipitate a salt of one enantiomer of CPA.

3. A process as claimed in claim 1 wherein the solvent is selected from the group consisting of an aliphatic, cycloaliphatic, aromatic hydrocarbon, hydroxylic solvent and any mixture thereof.

4. A process as claimed in claim 3 wherein the solvent is selected from the group consisting of methanol, ethanol, propanol, isopropanol, butanol, iso-butanol, tert-butanol, toluene and any mixture thereof.

5. A process as claimed in claim 4 wherein the solvent is selected from the group consisting of butanol, propanol, water and any mixture thereof.

6. A process as claimed in claim 1 wherein the amine resolving agent is an optically active amine.

7. A process as claimed in claim 6 wherein the optically active amine is an arylamine containing 8 to 20 carbon atoms.

8. A process as claimed in claim 7 wherein the arylamine is selected from the group consisting of α-phenyl-β-(p-tolyl)ethylamine, α-phenyl-β-phenylethylamine, α-phenylethylamine and N,N-dialkyl α-phenylethylamine.

9. A process as claimed in claim 7 wherein the arylamine is selected from the group consisting of N,N dimethyl, N,N diethyl, N,N dipropyl, N,N diisopropyl, N-methyl, N-ethyl and higher alkyl amines.

10. A process as claimed in claim 7 wherein the aryl amine is (S)(−)α-phenylethylamine.

11. A process as claimed in claim 1 wherein the amine resolving agent is used in amount of 0.4 to 0.65 mole per mole of (±)CPA.

12. A process as claimed in claim 1 wherein the amine is added in neat form or in the form of solution.

13. A process as claimed in claim 1 wherein the amine is added in one lot or over a period of time ranging from 10-60 minutes.

14. A process as claimed in claim 1 wherein the amine is added at a temperature in the range of 30 to 100° C.

15. A process as claimed in claim 1 wherein the amine is added to the racemic CPA solution.

16. A process as claimed in claim 1 wherein the racemic CPA solution is added to the amine.

17. A process as claimed in claim 1 wherein the solvent used is in the range of 20-40% as aqueous solution and two to three times by weight based upon the amount of CPA used.

18. A process as claimed in claim 1 wherein the resolution reaction is carried out over a period of 2 to 6 hours.

19. A process as claimed in claim 1 wherein the amine salt formed is substantially in the form of a precipitate.

20. A process as claimed in claim 1 wherein the temperature range during separation of optically active salt is in the range of ambient temperature to 80° C.

21. A process as claimed in claim 1 wherein the crystallized salt is separated by filtration or centrifugation.

22. A process as claimed in claim 1 wherein the optically active amine salt obtained is refined in a hydrophilic solvent selected from the group consisting of methanol ethanol propanol, isopropanol, butanol, 2-butanol, tert butanol and an aqueous mixture thereof.

23. A process as claimed in claim 22 wherein the hydrophilic solvent is selected from the group consisting of butanol, propanol and an aqueous mixture thereof.

24. A process as claimed in claim 1 wherein the optically active salt is refined at a temperature ranging from 40 to 120° C.

25. A process as claimed in claim 24 wherein the solvent used for refinement is in the range of 20-40% as aqueous solution and one to four times by weight based on the amount of optically active salt used.

26. A process as claimed in claim 24 wherein the duration of refinement is in the range of 3-5 hrs.

27. A process as claimed in claim 24 wherein the optically active salt is separated after refinement at a temperature in the range of 40 to 70° C.

28. A process as claimed in claim 24 wherein the optically active salt after refinement is separated by filtration or centrifugation.

29. A process as claimed in claim 1 wherein the optically active salt of (+) CPA after refinement is liberated using a mineral or an organic acid.

30. A process as claimed in claim 29 wherein the mineral acid used for liberation of optically active acid is selected from hydrochloric acid and sulphuric acid.

31. A process as claimed in claim 30 wherein the mineral acid is aqueous sulphuric acid.

32. A process as claimed in claim 29 wherein the aqueous mineral acid layer containing amine salt is combined with aqueous mineral acid layer obtained from recovery of the undesired (−) CPA.

33. A process as claimed in claim 1 wherein the mother liquor enriched with undesired (−) CPA salt obtained after precipitating the desired (+) CPA salt is concentrated at reduced pressure for recovery of (−) CPA.

34. A process as claimed in claim 1 wherein the undesired (−) CPA salt after concentration is treated with aqueous mineral/organic acids and extracted with hydrophilic/hydrophobic organic solvents and concentrated under reduced pressure for obtaining (−) CPA.

35. A process as claimed in claim 34 wherein the mineral acid used for liberation of (−) CPA from its amine salt is selected from hydrochloric acid and sulfuric acid.

36. A process as claimed in claim 35 wherein the mineral acid is aqueous sulfuric acid.

37. A process as claimed in claim 34 wherein the liberated acid is treated with an organic solvent selected from dichloromethane, dichloroethane, chloroform, toluene and hexane.

38. A process as claimed in claim 34 wherein the liberated acid is treated with an organic solvent comprising toluene.

39. A process as claimed in claim 34 wherein the aqueous mineral acid layer containing amine salt is combined with the corresponding aqueous mineral acid layer obtained from the liberation of desired (+) CPA to effect the recovery of optically active resolving agent.

40. A process as claimed in claim 39 aqueous mineral acid layers obtained from liberation of (+) CPA and (−) CPA are mixed, cooled preferably to 10 to 5° C. and extracted with aqueous caustic lye solution of concentration ranging from 20-80% to recover the resolving amine employed in resolution of (±) CPA.

41. A process as claimed in claim 40 wherein the concentration of the aqueous lye solution is in the range of 30-60%.

42. A process as claimed in claim 1 wherein the crude amine obtained is used in subsequent batches of (±) CPA and the alkaline layer is extracted with an hydrophilic/hydrophobic organic solvent selected from the group consisting of benzene, toluene, hexane, dichloromethane, dichloroethane and chloroform.

43. A process as claimed in claim 42 wherein the solvent is selected from benzene, toluene and hexane.

44. A process as claimed in claim 42 wherein the solvent is toluene.
